(19) United States
(12) Plant Patent Application Publication
(10) Pub. No.: US 2018/0139881 P1
(43) Pub. Date: May 17, 2018

(54) Tradescantia Plant Named 'Charlotte's Web'

(50) ;

(71) Applicant: Hans A. Hansen, Zeeland, MI (US)

(72) Inventor: Hans A. Hansen, Zeeland, MI (US)

(73) Assignee: Walters Gardens, Inc., Zeeland, MI (US)

(21) Appl. No.: 15/330,913

(22) Filed: Nov. 14, 2016

(51) Int. Cl. A01H 5/02

(52) U.S. Cl. PLT/263.1

(57) ABSTRACT

A new and distinct plant cultivar of winter-hardy spiderwort named Tradescantia ‘Charlotte's Web’ characterized by lanceolate foliage of gold coloring accented by thin red margins in the spring and late season. The flowers are soft blue over a 22 week period beginning early June and continuing through hard frost. The new plant is suitable for landscaping as a specimen or en masse.

Botanical classification: Tradescantia hybrid.

Varietal denomination: ‘Charlotte's Web’.

### BACKGROUND OF THE PLANT

The present invention relates to a new and distinct spiderwort plant, Tradescantia ‘Charlotte's Web’ hereinafter also referred to as the new plant or just the cultivar name, ‘Charlotte's Web’. The new plant was hybridized in a greenhouse at a wholesale perennial nursery in Zeeland, Mich., USA on Apr. 25, 2012 by the inventor as a cross between the proprietary unreleased female or seed parent which was a cross between ‘Sweet Kate’ (not patented) and ‘Tall and Blue’ (not patented) later assigned the breeder code H10-03-01 and the male or pollen parent was a proprietary unreleased plant assigned the breeder code 11-SRF-307, which was a chemically induced mutation from ‘Zwanenburg Blue’ (not patented). The new plant was assigned the breeder code 12-6-10 when it passed the first evaluation phase in summer of 2013. The new plant has been asexually propagated by stem cuttings, node cuttings and division at the same nursery in Zeeland, Mich., USA since 2013 with the resultant asexually propagated plants having retained all the same traits as the original plant. ‘Charlotte's Web’ is stable and reproduces true to type in successive generations of asexual reproduction.

No plants of Tradescantia ‘Charlotte's Web’ have been sold in this country or anywhere in the world prior to the filing of this application, nor has any disclosure of the new plant been made prior to the filing of this application with the exception of that which may have been disclosed within one year of the filing of this application and was either derived directly or indirectly from the inventor.

### SUMMARY OF THE INVENTION

The new Tradescantia variety of the present invention is a member of the Commelinaceae family and passed initial evaluation in the summer of 2013 as a seedling growing in the trial garden of a wholesale perennial nursery in Zeeland, Mich., U.S.A. among several other seedlings of the same cross and other crosses.

The nearest comparison varieties are ‘Sweet Kate’ (not patented) and ‘Radtrad’ U.S. Plant Pat. No. 24,733. The new variety can be readily distinguished from ‘Sweet Kate’ in that the new plant is about 50% taller, is less prone to scorching in bright sun, has attractively distinctive red marking on the yellow shoots as it emerges in the spring and has a much longer flowering season. Compared to ‘Radtrad’ the new plant is less prone to sun scorch when grown in full-sun conditions and develops a wider plant faster than ‘Radtrad’.

The following traits of Tradescantia ‘Charlotte's Web’ in combination distinguish the new plant from all other spiderworts known to the inventor:
1. Lanceolate foliage emerges in the spring with thin red margin on chartreuse foliage.
2. Foliage later develops into nice gold color with strong resistance to burning in direct sun.
3. Long leaves set off flowers of soft blue over a prolonged season beginning in late May in Michigan and continuing until mid-fall.

### BRIEF DESCRIPTION OF THE DRAWINGS

The photographs of the new plant demonstrates the overall appearance and unique traits of ‘Charlotte's Web’ as a two-year-old plant grown in a full-sun trial garden in Zeeland, Mich. with supplemental water and fertilizer provided as needed. The colors are as accurate as reasonably possible with color reproductions. Variation in ambient light spectrum, source and direction may cause the appearance of minor variation in color.

FIG. 1 shows a close-up of the buds and flowers.

FIG. 2 shows a close-up of the foliage with thin red margin

FIG. 3 shows the plant in the landscape in mid-summer.

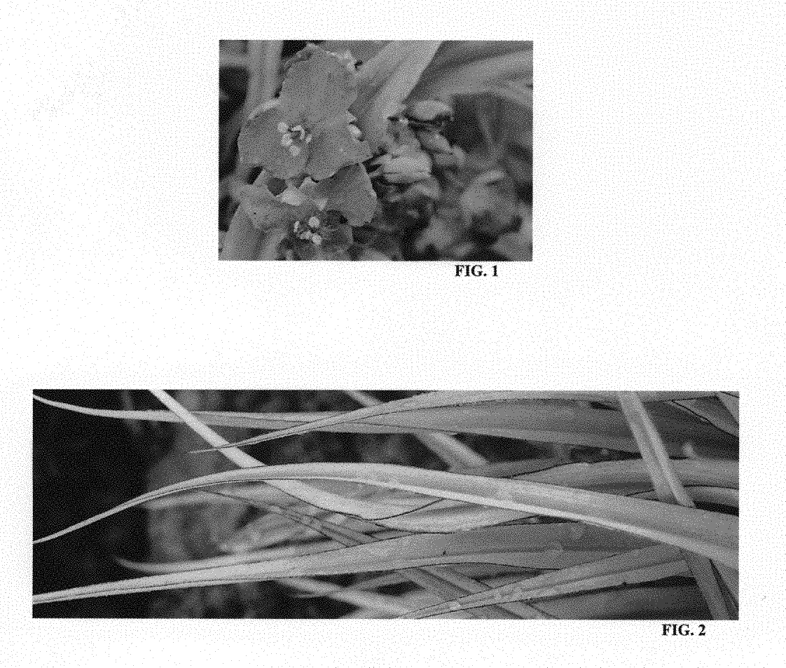

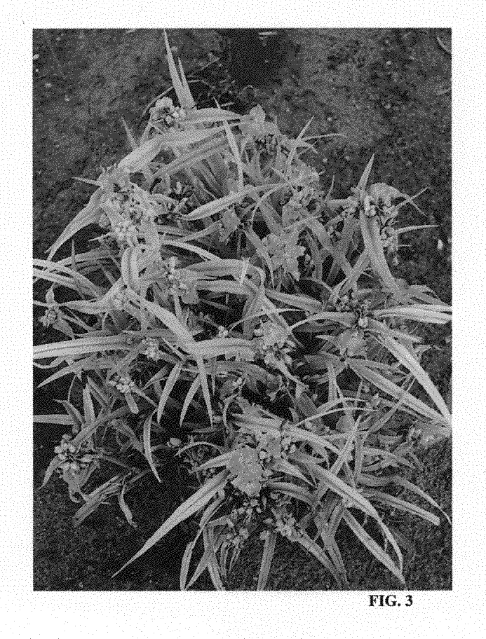

### DETAILED BOTANICAL DESCRIPTION

The following detailed descriptions and color references of the new cultivar are based on the 2001 edition of The Royal Horticultural Colour Chart except where common dictionary terms are used. The plant described is a two-year-old plant grown in a full-sun trial garden in Zeeland, Mich. with supplemental water and fertilizer. Tradescantia ‘Charlotte's Web’ has not been observed under all possible environments. The phenotype may vary slightly with different growing environments such as temperature, light, fertility, soil pH, moisture and maturity levels, but without any change in the genotype.
Botanical classification: Tradescantia hybrid.
Parentage: female (seed parent) internal breeder code H10-03-01; male (pollen parent) internal breeder code 11-SRF-307.
Plant habit: arching bushy mound, winter-hardy herbaceous perennial.
Size: about 45.0 cm tall and about 80.0 to 90.0 cm across including foliage and flowers.
Leaves: sessile; alternate; linear; conduplicate; arching; slightly glaucous abaxial and adaxial; adaxial puberulent along about 1.0 mm of margin; micro-serrulate margin; narrowly acute apex and decurrent base; up to about 39.0 cm long and about 19.0 cm across.
Leaf color: abaxial and adaxial margin of emerging leaves nearest RHS 187A; variable with light intensity, both surfaces between RHS 138A and RHS 137D in lower light and nearest RHS 11A in bright light; in cooler weather margin becomes nearest RHS 187B.
Venation: parallel; young expanding leaves abaxial midrib puberulent to pubescent; mature leaves adaxial and abaxial midrib and secondary veins glabrous.
Vein color: midrib and secondary veins same as surrounding leaf tissue on abaxial and adaxial.
Petiole: absent.
Inflorescence: terminal umbel-like cyme; with 12 to 15 flowers.
Flowering season: June to November for up to 22 weeks.
Peduncle: absent.
Pedicels: terete; outright; glabrous; about 15.0 mm long and about 1.0 mm diameter.
Pedicel color: base nearest RHS 157D and distally nearest RHS 59B.
Buds: partially flattened ovoid; about 10.0 mm long and 3.5 mm wide.
Bud color: exposed petals nearest RHS N92D and sepals nearest RHS 146D.
Flower: single; perfect; actinomorphic; hypogynous; to about 30.0 mm across and about 10.0 mm deep; no fragrance observed.
Flower lastingness: about one day.
Petals: three; broadly acute apex, cuneate base and entire margin; glabrous both adaxial and abaxial; about 14.0 mm long and 14.0 mm across.
Petal color: adaxial between RHS 93B and RHS 93C with the adaxial base between RHS 93C and RHS 93D; abaxial apex and center between RHS 93B and RHS 93D with base nearest RHS 92C.
Sepal: typically three; ovate; concavo-convex; acute apex; truncate base; margins entire; adaxial surfaces glabrous; abaxial surface puberulent to pubescent; about 7.5 mm long and about 4.0 mm across.
Sepal color: typically three per flower; adaxial nearest THR N144A with some speckles of nearest RHS 59A; abaxial nearest RHS N144A.
Androecium: stamen six:
   Filament.—six, terete; about 5.0 mm long and about 0.3 mm diameter; covered in hairs about 4.0 mm long by 0.1 mm diameter; color nearest RHS 79C.
   Anther.—ovoid; basifixed; longitudinal; about 0.5 mm long and about 0.3 mm across; color nearest RHS 9A.
   Pollen.—abundant; nearest RHS 13A.
   Gynoecium.—single.
   Style.—terete; about 6.0 mm long and 0.3 mm diameter; color nearest 85D proximally, between RHS N88A and RHS N89B distally.
   Ovary.—superior; about 3.0 mm long and 1.9 mm diameter; color between 145D and RHS 150D.
Fruit and seeds: none observed.
Stem: terete; mostly upright; about 47 cm long and 8.0 mm diameter at base.
Stem color: between RHS 147C and RHS 147D; can develop heavy tinting of RHS 187B with direct sun and cooler temperatures.
Node: typically swollen above to about 11.0 mm diameter; average internode 9.4 cm long.
Node color: typically same as surrounding stem.
Resistance to diseases: The new plant shows strong resistance to foliage burning in direct sun compared with other yellow-foliaged spiderwmt plants. Other resistance beyond that which is typical for spiderwort has not been observed. Tradescantia ‘Charlotte's Web’ is winter hardy to at least USDA zone 3.

It is claimed:

1. A new and distinct cultivar of a Tradescantia hybrid plant named ‘Charlotte's Web’ substantially as herein illustrated and described.
